(19) United States
(12) Patent Application Publication
(10) Pub. No.: US 2021/0121122 A1
(43) Pub. Date: Apr. 29, 2021

(54) Quantification of Liver Steatosis from a biopsy using a Computer Imaging Platform

(71) Applicant: The Board of Trustrees of the Leland Stanford Junior University, Stanford, CA (US)

(72) Inventors: Marc L Melcher, Menlo Park, CA (US); Linfeng Yang, Beijing (CN); Raja R. Narayan, Mountain View, CA (US); Simon Chen, Los Altos, CA (US); Natasha Abadilla, East Palo Alto, CA (US)

(21) Appl. No.: 17/075,535

(22) Filed: Oct. 20, 2020

Related U.S. Application Data
(60) Provisional application No. 62/926,453, filed on Oct. 26, 2019.

(51) Int. Cl. A61B 5/00

(52) U.S. Cl. CPC A61B 5/4244; A61B 5/7264; A61B 5/742; A61B 5/7275

(57) ABSTRACT

A method and (portable) device are provided for quantifying liver steatosis. The quantitative assessment of liver steatosis predicts whether a liver from the liver transplant donor is suitable for transplantation. Specifically, the quantitative assessment of liver steatosis predicts an associated risk for early allograft dysfunction. A biopsy image from a liver transplant donor is obtained from an automatic quantitative assessment of liver steatosis is made using a pre-trained artificial intelligence algorithm for identifying parameters of liver steatosis. The biopsy image is input to the pre-trained artificial intelligence algorithm. The quantitative assessment of liver steatosis is the output of the pre-trained artificial intelligence algorithm. Embodiments provide for rapidly diagnosing potential donor liver allografts at the time of procurement with a point-of-care device deployed with the transplant surgery team.

### CROSS-REFERENCE TO RELATED APPLICATIONS

This application claims priority from U.S. Provisional Patent Application 62/926,453 filed Oct. 26, 2019, which is incorporated herein by reference.

### FIELD OF THE INVENTION

This invention relates to use of a portable device to perform rapid histopathological assessments of tissue, especially for liver tissue.

### BACKGROUND OF THE INVENTION

When surgeons perform a biopsy, they often need a relatively rapid histopathological assessment of the tissue. In the field of liver transplantation, this is particularly relevant. Despite the many patients in need of liver transplantation and the limited supply of organ donors, about 10% of procured livers could be discarded yearly, many based on histologic findings of donor liver biopsies. Fat involvement, or hepatic steatosis of the liver, is a frequent finding among liver donors and generally thought to be associated with early graft failure after transplantation. Hepatic steatosis as a cause for liver failure is in fact projected to be the most common indication for liver transplantation in the coming decade, making it a relatively common finding among eligible liver donors. Currently, the quantification of steatosis is highly variable even among trained pathologists. A wide range (30-70%) is reported of acceptable degrees of steatosis measured as the percentage of fat detected in biopsies of the liver for a transplantable liver.

Transplantologists are concerned that this wide range is actually a consequence of variability associated with the process of quantifying liver steatosis. Further, transplantologists fear that eligible liver recipients are missing out on viable liver grafts and even of greater concern, that adequate grafts are wrongly discarded.

### SUMMARY OF THE INVENTION

The present invention provides a method and (portable) device for quantifying liver steatosis. In one embodiment, the method and device provide a quantitative assessment of liver steatosis which predicts whether a liver from the liver transplant donor is suitable for transplantation. The quantitative assessment of liver steatosis predicts an associated risk for early allograft dysfunction.

A biopsy image from a liver transplant donor is obtained from an automatic quantitative assessment of liver steatosis is made using a pre-trained artificial intelligence algorithm for identifying parameters of liver steatosis. The biopsy image is input to the pre-trained artificial intelligence algorithm. The quantitative assessment of liver steatosis is the output of the pre-trained artificial intelligence algorithm. The automatic determination of the quantitative assessment of liver steatosis using a pre-trained artificial intelligence algorithm can be embedded on a computer processing chip.

The quantitative assessment of liver steatosis can be expressed as a percentage of liver steatosis. The quantitative assessment of liver steatosis can be displayed on a display of the (portable) device. The parameters of liver steatosis are fat vesicles. The method and device can also be used or have the potential to be updated in iterations to identify other inflammatory features such as fibrosis and hepatocyte ballooning.

Embodiments of the invention provide the advantages of rapidly diagnosing potential donor liver allografts at the time of procurement with a point-of-care device deployed with the transplant surgery team. Rapid diagnosis limits cold ischemia time of the donor liver therefore improving the early postoperative as well as long term outcomes for the liver recipient. Additionally, use of an accessible, point-of-care, computer vision-based platform enables objective quantification of donor liver steatosis in real-time. The current standard of care calls for a pathologist at a local community hospital to review a biopsy of the potential donor liver, often spontaneously or after office hours. This device would circumvent the delay and subjectivity inseparably linked to this current standard.

Embodiments of the invention have the advantages that it is portable, relatively fast compared to current methods, the assessment is more reproducible than when performed by human pathologists, and correlates better with early allograft outcome in liver transplantation.

### BRIEF DESCRIPTION OF THE DRAWINGS

FIG. 1 shows according to an exemplary embodiment of the invention the method of quantifying liver steatosis and based on that predicting whether a liver is suitable for transplantation.

FIG. 2 shows according to an exemplary embodiment of the invention annotation of steatosis on digital donor liver biopsy slides by the computer vision artificial intelligence platforms. FCN=Fully convolutional networks, U-Net=U-shaped up-sampling networks.

FIG. 3 shows according to an exemplary embodiment of the invention mean intersection over union for prediction of donor liver steatosis by computer vision artificial intelligence platforms with varying number of training tiles. FCN=Fully convolutional networks, S-U-Net=Shallow u-shaped up-sampling networks, U-Net=u-shaped up-sampling networks, IU=intersection over union.

FIG. 4 shows according to an exemplary embodiment of the invention a comparison of steatosis scored by pathologists and the U-Net computer vision artificial intelligence platform.

FIG. 5 shows a schematic of the device implementing the method for quantification of liver steatosis according to an exemplary embodiment of the invention.

FIG. 6 shows a CAD model of the device implementing the method for quantification of liver steatosis according to an exemplary embodiment of the invention.

FIG. 7 shows a Computer Vision Artificial Intelligence (CVAI) Model model according to an exemplary embodiment of the invention.

### DETAILED DESCRIPTION

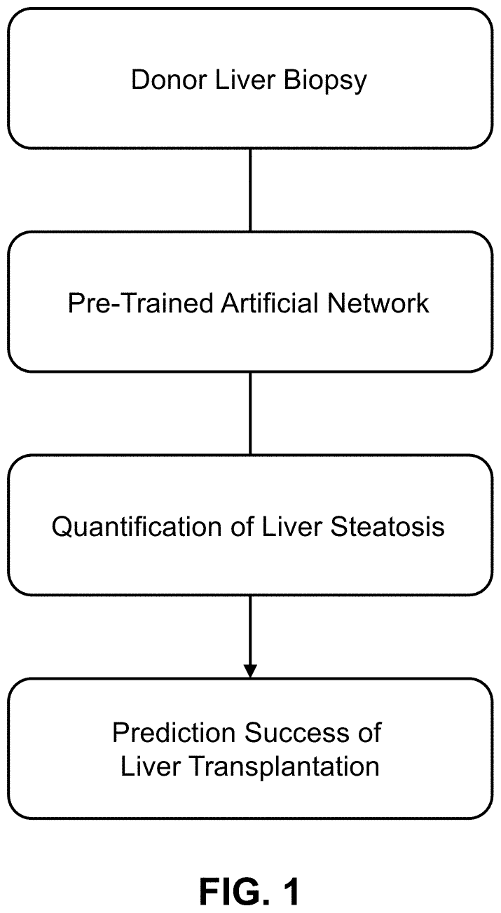

A method and device are provided to score/quantify hepatic steatosis to predict the associated risk for early allograft dysfunction (EAD) (FIG. 1).

### Patient and Tissue Selection

All donor liver biopsies performed over a period of 5 years were identified from a prospectively-maintained liver histopathology database. Patients were excluded if there was inadequate liver tissue for histopathologic review or digitization and if the date of the last follow-up was less than 60 days. Recorded information regarding donor and recipient characteristics, surgical history, histopathology, perioperative outcomes, follow up, and survival were collected. The Model for End-Stage Liver Disease (MELD) score for recipients with cancer was not adjusted for tumor burden.

All slides of donor liver biopsies were prepared with hematoxylin and eosin staining, re-reviewed in consensus by two independent pathologists experienced in analyzing donor liver graft tissue (SBC and JPH), and digitized. The pathologists and the CVAI platforms scored the slides for the percent area of total steatosis out of the total hepatic parenchyma.

### Constructing Computer Vision Artificial Intelligence (CVAI) Models

Four CVAI models were developed incorporating computer vision and convolutional neural networks constructed with varying network depths to label fat globules: fully convolutional networks of 5 dimensions (FCN 5), 10 dimensions (FCN 10), U-shaped up-sampling networks (U-Net), and Shallow U-Net. The digital slides were partitioned into tiles with the background excluded. The tiles were divided into two groups with a 1:99 split for a training set with a resolution of 256×256 pixels and a prediction set with a resolution of 512×512 pixels. The digitized tiles with annotated fat globules served as the ground truth to train the four CVAI models. The precision of steatosis scoring was based on this ground truth and measured using the mean intersection over union (IU) using 45, 90, and 181 tiles for training. The CVAI models then processed the remaining unlabeled, digitized slides calculating the percent steatosis.

### Defining Post-Transplantation Outcomes

The diagnosis of EAD was defined as either total bilirubin≥10 mg/dL or INR≥1.6 on the seventh day following transplantation or either AST or ALT>2000 U/L within the first 7 days after transplantation. The condition of the patient 60 days following transplantation was recorded specifying whether re-transplantation was necessary or if the patient was deceased.

### Statistical Analysis

The relationship of EAD and 60-day mortality to histopathologically and CVAI-scored steatosis was investigated using the Wilcoxon Rank-Sum test. Univariate logistic regression analysis was performed to identify pre-transplantation factors associated with EAD considering P<0.1 to be significant enough to include in the multivariate analysis where two-sided P values <0.05 were considered statistically significant. All statistical analyses were performed using R version 4.0.2 (R Foundation for Statistical Computing, Vienna, Austria).

### Fidelity of Computer Vision Artificial Intelligence Models

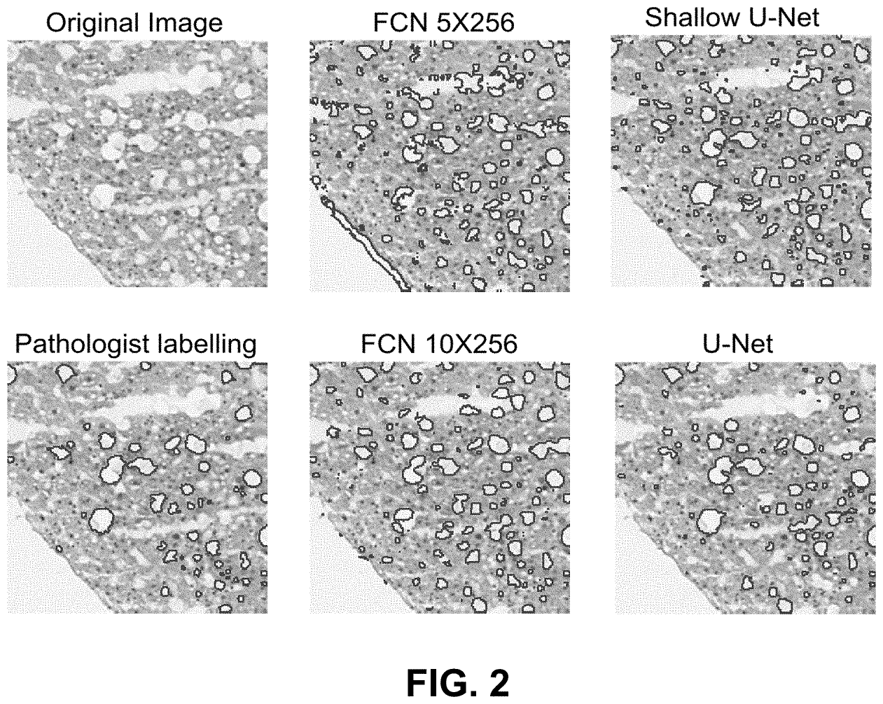

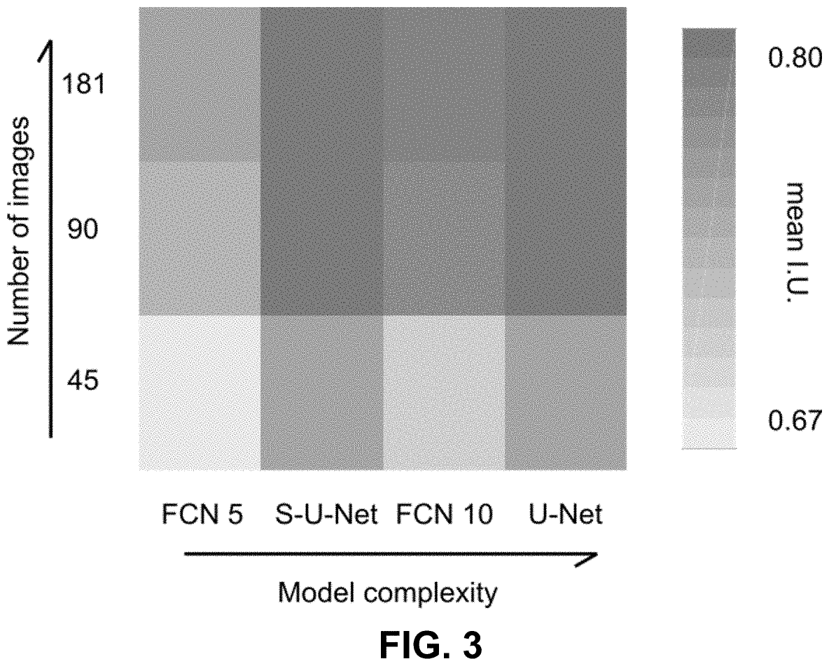

From 91 digitized donor liver slides, 25,494 tiles were generated of which 181 were used to train the CVAI platforms. Relative to the ground truth of pathologist labeling, the CVAI platforms were able to identify fat globules with varying degrees of success (FIG. 2). The U-Net and Shallow U-Net platforms had the highest performance (mean IU=0.80 and 0.80, respectively) when compared to pathologist labeling (FIG. 3). Optimal performance could be achieved by the U-Net and Shallow U-Net platforms with half (mean IU=0.80 and 0.80, respectively) and a quarter the number of tiles (mean IU=0.75 and 0.75, respectively).

### Prediction of Steatosis and Outcomes by Pathologists and Artificial Intelligence

The median steatosis score estimated by the average of the pathologists (20%, range=0-100%) was significantly greater than the median steatosis score estimated by U-Net, the optimal CVAI platform (3%, range=0.2-19.3%, P<0.001). Of the 90 donor livers, 41 (46%) were transplanted at our institution. The median age for the donors and recipients was 50 and 57 years old, respectively, and 18 (45%) recipients were female. The median steatosis on histopathologic review of transplanted donor livers was 15% (range=0-95%) and 2% (range=0.2-14%, P<0.001) per CVAI. Among the recipients, 19 (46%) developed EAD and had higher median steatosis (30%, range=0-95%) on histopathologic review than CVAI calculation (3%, range=1-12%, P<0.001). Compared to those that developed EAD, the donor livers of the remaining 23 recipients did not differ significantly on histopathologic review with a median steatosis of 10% (range=0-90%, P=0.056); however, the CVAI calculated median steatosis was significantly lower than the EAD patients at 2% (range=0.2-14%, P=0.020).

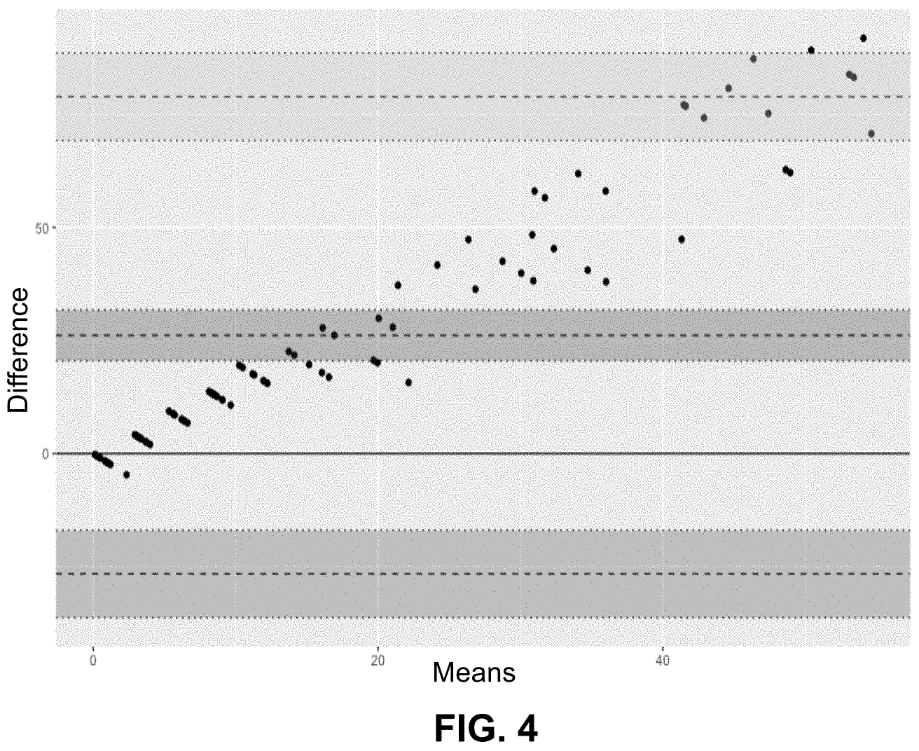

When charting the steatosis score from histopathologic review or the CVAI platform by increasing CVAI steatosis score, notably divergent scores were observed when pathologists were reviewing slides they deemed to have 20% or greater total steatosis (FIG. 4).

### Logistic Regression Analysis of Post-Transplantation Outcomes

On univariate logistic regression analysis (Table 1), histopathologic total steatosis score was not associated with EAD (OR=0.99, 95% CI=0.93-1.06, P=0.835). A multivariate logistic regression model, which included all significantly associated factors, was constructed and revealed that only CVAI total steatosis score (OR=1.50, 95% CI=1.14-2.27, P=0.014) was independently associated with EAD.

TABLE 1
Logistic regression analysis of pre-transplantation factors
associated with early allograft dysfunction.
MELD = Model for End-Stage Liver Disease,
AI = Artificial intelligence, 95% CI = 95% confidence interval.

| | Univariate | | | Multivariate | | |
|---|---|---|---|---|---|---|
| Variables | OR | 95% CI | P | OR | 95% CI | P |
| Donor age | 1.02 | 0.93-1.13 | 0.710 | | | |
| Donor sex | 0.11 | <0.01-2.04 | 0.161 | | | |
| Donor BMI | 0.90 | 0.72-1.07 | 0.269 | | | |
| Donor Diabetes | 3.14 | 0.83-17.33 | 0.125 | 2.24 | 0.95-6.36 | 0.088 |
| Donor Cause | 1.20 | 0.53-2.67 | 0.637 | | | |
| of Death | | | | | | |
| Recipient age | 0.97 | 0.86-1.07 | 0.531 | | | |
| Recipient sex | 0.54 | 0.05-4.97 | 0.577 | | | |
| Recipient BMI | 0.92 | 0.71-1.16 | 0.523 | | | |
| Recipient | 0.03 | <0.01-0.80 | 0.081 | 0.08 | <0.01-1.01 | 0.096 |
| Diabetes | | | | | | |
| Recipient Cause | 0.67 | 0.22-1.51 | 0.378 | | | |
| of Liver Failure | | | | | | |
| Recipient MELD | 0.81 | 0.60-0.98 | 0.070 | 0.89 | 0.76-1.02 | 0.108 |
| Cold ischemia | 1.00 | 0.99-1.02 | 0.702 | | | |
| time | | | | | | |
| Path steatosis | 0.99 | 0.93-1.06 | 0.835 | | | |
| CVAI steatosis | 1.88 | 1.13-4.04 | 0.045 | 1.50 | 1.14-2.27 | 0.014 |

### CVAI Platform

Poor histopathology is a common reason for discarding otherwise viable donor livers. The most common reason for non-utilization of procured grafts was fatty liver disease, noted in over a third of discarded donor livers. In one embodiment of the invention, a high fidelity CVAI platform was developed capable of calculating donor liver steatosis and discerning livers at risk for EAD.

The CVAI platforms are convolutional neural networks that employ deep learning models using computer vision to segment images for features of interest. These platforms could be trained with relatively few slides since each slide contained many examples of the feature being trained for—lipid droplets. Among the platforms, the Shallow U-Net and U-Net models were able to predict steatosis scores among the training set images with high fidelity utilizing as few as 90 tiles. In the prediction set, the median steatosis score estimated by the U-Net CVAI platform was over a fifth lower than the score estimated by pathologists and five times lower in the subset of donor livers that were transplanted. At the institution of the inventors, the cutoff for permitting transplantation of an otherwise optimal donor liver with isolated hepatic steatosis is approximately 60%. Despite correctly labeling the fat content of donor biopsies in the training set, assignment of higher steatosis scores by the pathologist may reflect an overestimation of the percent labeled area relative to the rest of the hepatic parenchyma. However, it also may signal other characteristics on the slide that raise concerns for the quality of the graft (e.g. fibrosis, hepatocyte ballooning). As such, the findings in this invention suggest that the actual steatosis scores may be lower than what pathologists have been reporting.

Most pathologists provide an estimate for the steatosis score rather than calculate the total steatosis area over the area of hepatic parenchyma. This practice can lead to a visual perception illusion, a phenomenon described for its association with bile duct injury during laparoscopic cholecystectomy, wherein the true steatosis score is upscaled because of the confounding presence of artifactual cracks in the tissue and the background that bears similar color to the fat vacuoles. With the increasing accessibility of deep learning platforms with computational tools, the performance of menial tasks such as scoring total steatosis or counting numerous mitoses should be reserved for automated algorithms. As a result, the role of the pathologist in the 21st century is shifting from rote repetitive tasks to providing higher-level insight on histopathologic features incorporating the broader clinical picture.

Nearly half of transplanted donor livers developed EAD within the first week of transplantation. With the rare occurrence of graft failure, re-transplantation, or death after transplantation, an intermediate surrogate outcome like EAD is necessary to identify risk factors associated with donor liver grafts. It has demonstrated that EAD noted early in the post-transplantation course is associated with renal failure at discharge, graft loss within a year, and death.

Among the transplanted, the CVAI platform could discern which donor livers developed EAD, however, the histopathologic scoring could not. Although the difference in the steatosis scores between the grafts developing EAD and those that did not was statistically significant, a clinically significant difference could not be perceived. Interestingly, the median steatosis score for the grafts that developed EAD was within the range that our institution would consider transplantable. This may reflect a spurious finding from an underpowered survey limited by the selection bias of reviewing slides from donor livers that were permitted for transplantation in the first place. If training platforms with knowledge of which donor livers went on to develop EAD, nuanced features detectable by deep learning segmentation may enable better stratification of grafts at risk for poor outcomes. For example, features detected from segmented diagnostic imaging have been reported to be associated with tumor genotypes, response to chemotherapy, and risk of post-hepatectomy liver insufficiency. Characterization of these features may result in better stratification of donor grafts associated with EAD or long term poor outcomes.

Furthermore, CVAI steatosis score was the only factor independently associated with EAD on both univariate and multivariate analysis. Interestingly the CVAI steatosis score, but not the histopathologic score, was independently associated with EAD. This encourages further investigation into whether CVAI platforms can better characterize other histopathologic findings critical to note before proceeding with transplantation, such as fibrosis or hepatocyte ballooning.

### CVAI Example

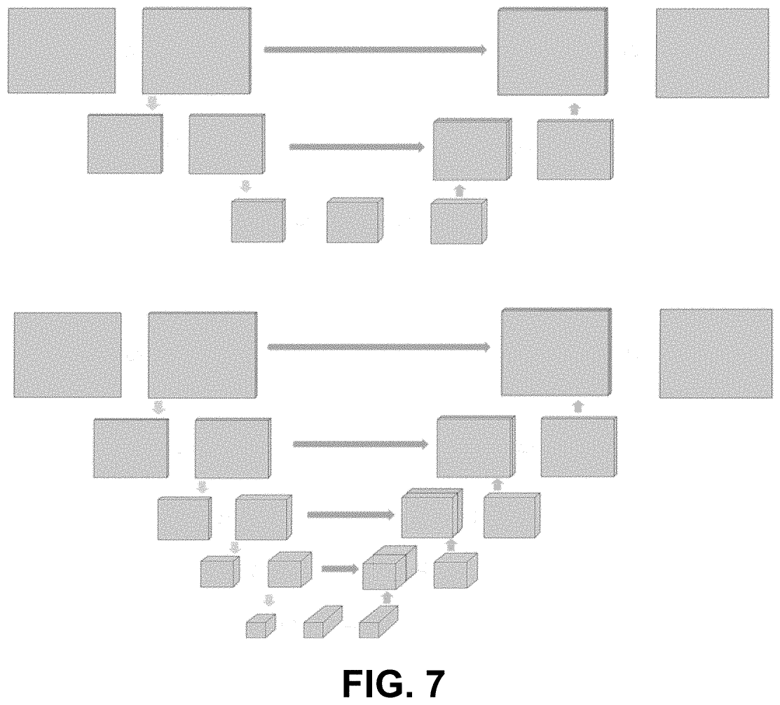

FIG. 7 shows an example of the shallow UNET and standard UNET. The network alogrithm is based on the work described in PLoS Comput Biol. 2020 September; 16(9): e1008193. Published online 2020 Sep. 14. doi: 10.1371/journal.pcbi.1008193 PMCID: PMC7515182 PMID: 32925919 NuSeT: A deep learning tool for reliably separating and analyzing crowded cells.

### Portable Device

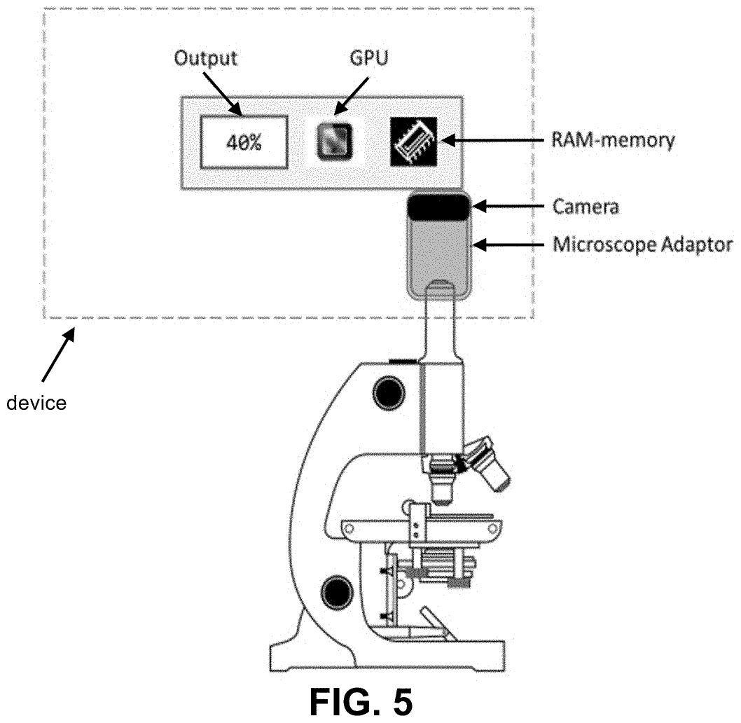

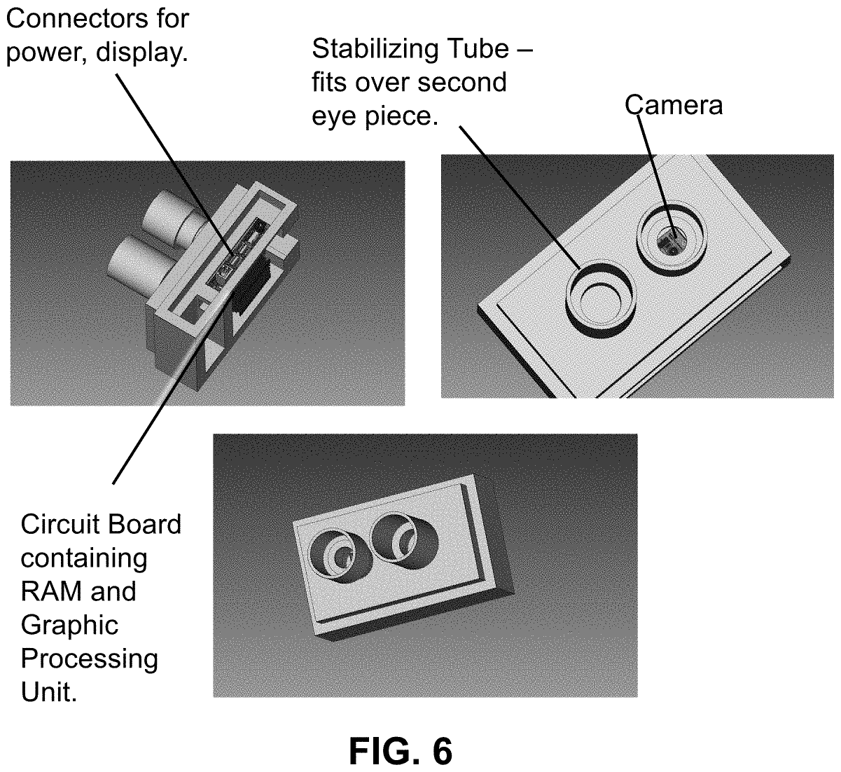

In one example, the invention can be embodied as a (trainable) portable device that can scan a slide through a microscope (FIGS. 5 and 6). Then by using a pretrained artificial intelligence model, the biopsy can be assessed. In one example, the device includes a microscope eye piece adaptor, a high-resolution digital camera, a graphics processing unit (GPU), Random Access Memory (RAM), a small display, and an embedded AI algorithm. The AI processing algorithm is pre-trained for specific tasks and is loaded/embedded into the device. The AI algorithm is specifically developed to recognize fat globules within a liver biopsy because it has been shown that donor livers with high-fat content tend to do less well than donors with no fat content.

The device proposed in this invention is able to photograph the specimen through a microscope. The image is stored on the device and processed using the specific AI algorithm. The process is done locally to address privacy concerns. It is not done in the cloud. The AI algorithm calculates the percentage of fat seen on the slide and reports the percentage to the output display.

The AI algorithm was trained using biopsies from livers being considered for donation. The algorithm can identify with high specificity and sensitivity the fat globules within the liver tissue. Then it can calculate the percentage of fat (steatosis) within the liver.

In another example, the invention can be embodied as a (trainable) portable device that can enable point-of-care, rapid diagnosis of hepatic steatosis. This would circumvent the need for a community pathology potentially inexperienced with liver histopathology being called in to score a biopsy of the donor liver for steatosis. This currently is the standard of care and adds to the cold ischemia time of the donor liver allograft which has been shown to increase the risk of post-transplantation complications.

What is claimed is:

1. A method for quantifying liver steatosis, comprising:
(a) obtaining a biopsy image from a liver transplant donor; and
(b) automatically determining a quantitative assessment of liver steatosis using a pre-trained artificial intelligence algorithm for identifying parameters of liver steatosis, wherein the biopsy image is input to the pre-trained artificial intelligence algorithm, and wherein a quantitative assessment of liver steatosis is output of the pre-trained artificial intelligence algorithm.

2. The method as set forth in claim 1, wherein the quantitative assessment of liver steatosis predicts whether a liver from the liver transplant donor is suitable for transplantation.

3. The method as set forth in claim 1, wherein the quantitative assessment of liver steatosis predicts an associated risk for early allograft dysfunction.

4. The method as set forth in claim 1, wherein the quantitative assessment of liver steatosis is a percentage of liver steatosis.

5. The method as set forth in claim 1, wherein the parameters of liver steatosis are fat vesicles.

6. The method as set forth in claim 1, wherein the step of automatically determining a quantitative assessment of liver steatosis using a pre-trained artificial intelligence algorithm is embedded on a computer processing chip.

7. The method as set forth in claim 1, further comprising displaying the quantitative assessment of liver steatosis.

8. The method as set forth in claim 1, wherein the method for quantifying liver steatosis is embodied as a single portable device.
